(19) United States
(12) Patent Application Publication
(10) Pub. No.: US 2012/0158027 A1
(43) Pub. Date: Jun. 21, 2012

(54) CAPSULOTOMY METHODS AND APPARATUS USING HEAT

(76) Inventors: Ala Moradian, St. Louis, MO (US); Brian McCary, Clayton, MO (US)

(21) Appl. No.: 12/969,670

(22) Filed: Dec. 16, 2010

(51) Int. Cl. A61F 9/007

(52) U.S. Cl. 606/166

(57) ABSTRACT

An injector for performing a capsulotomy on a capsular bag, comprising an injector body, an injector nozzle coupled to the injector body, the injector nozzle having a lumen extending therethrough ending in an open tip, a strand of shape-memory material adapted to achieve a predetermined shape when ejected from a tip through an opening, a heating unit coupled to the strand to cause the strand to achieve an elevated temperature.

### FIELD OF INVENTION

The present invention relates to capsulotomy methods and apparatus, and more particularly to capsulotomy methods and apparatus using heat.

### BACKGROUND OF THE INVENTION

Surgical procedures for replacing a crystalline lens of a human eye are well known. To practice such procedures, typically an incision is made in a patient's cornea or sclera, and an opening is made in the anterior portion of the capsular bag which surrounds the patient's lens. Such an opening is commonly referred to as a “capsularhexis” (or simply as a “rhexis”); and the process by which the opening is made is commonly referred to as a “capsulotomy.” After the opening is made, the eye's lens is removed through the rhexis and a replacement lens (referred to herein as an intraocular lens (or an IOL)) is inserted into the capsular bag through the rhexis.

According to conventional procedures, the incision in the cornea is made with a scalpel, and a pointed stylet or needle is inserted through the incision and is used to rip or tear a generally circular rhexis into the anterior capsular bag.

The use of such a stylet or needle to tear an opening in the anterior capsular bag requires a steady hand and a very high degree of skill. Even when practiced by a skilled surgeon, the potential for incorrectly performing the procedure is substantial.

Forming a rhexis having a larger or smaller diameter than that desired, or inadvertently tearing the capsular bag may prevent the remaining capsular bag from properly retaining an IOL and may result in complications, including infection, damage to other portions of the eye, and potential loss of sight.

As such, although conventional capsulotomy procedures have proven generally suitable for lens replacement surgery, it has deficiencies which may detract substantially from its safety and efficacy. In view of these shortcomings, it is highly desirable to provide a means for reliably and safely forming a rhexis of a desired diameter and shape.

### SUMMARY

Aspects of the present invention are directed to an injector for performing a capsulotomy on a capsular bag, comprising an injector body, an injector nozzle coupled to the injector body, the injector nozzle having a lumen extending therethrough ending in a tip having an opening, a strand of shape-memory material adapted to achieve a predetermined shape when ejected from through opening, and a heating unit coupled to the strand to cause the strand to achieve an elevated temperature.

In some embodiments, the injector further comprises an actuation apparatus for causing the strand to be ejected through the opening. The tip may be adapted to fit through an incision in the eye of less than 3 mm. The strand may be adapted to form a closed shape or substantially closed shape upon ejection through the opening.

In some embodiments, the shape-memory material comprises nitinol. The heating unit may comprise a monopolar heating system. In some embodiments, the strand is provided with a sharp tip to facilitate cutting of the capsular bag. The injector may comprise a second strand of shape-memory material.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1A is a schematic side view illustration of an example of an injector for performing capsulotomies according to aspects of the present invention in which a strand of shape-memory material is retracted within the injector;

FIG. 1B is a schematic side view illustration of the injector shown in FIG. 1A in which the strand of shape-memory material has been ejected from the nozzle of the injector;

FIG. 1C is a schematic top view illustration of the injector shown in FIG. 1A in which the strand of shape-memory material has been ejected from the nozzle of the injector;

FIG. 2 is a schematic, partial, top view illustration of the injector shown in FIG. 1A in which the strand of shape-memory material has been ejected from the nozzle of the injector into an eye on which a capsulotomy is to be performed; and

FIG. 3 is a schematic, partial, side view illustration of a second example of an injector for performing capsulotomies according to aspects of the present invention in which two strands of shape-memory material have been ejected from the nozzle of the injector, one strand on an exterior surface of the capsular bag and one strand on an interior surface of the capsular bag.

### DETAILED DESCRIPTION

Aspects of the present invention are directed towards an injector for performing capsulotomies in which a strand of shape-memory material is inserted into an incision in the eye and used to heat a surface of the capsular bag of the eye. The tissue that is heated is weakened along a tear line such that a subsequent force applied to the capsular bag can cause a rhexis to be formed in a predetermined shape along the tear line.

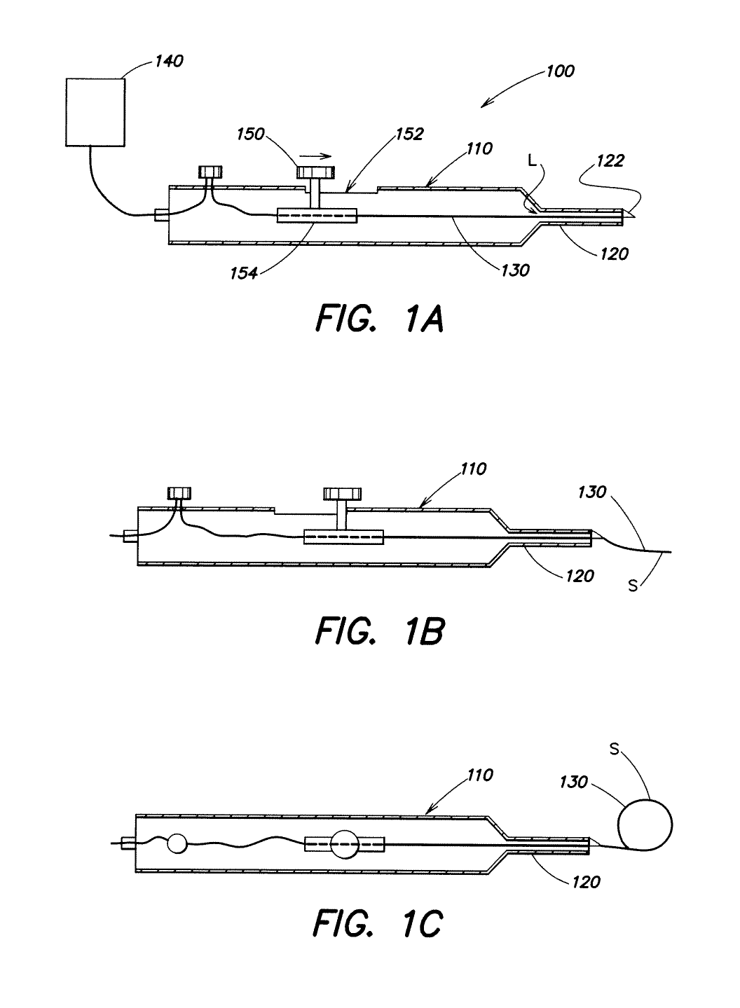

FIG. 1A is a schematic side view illustration of an example of an injector 100 for performing capsulotomies according to aspects of the present invention in which a strand of shape-memory material 130 is retracted within the injector. Injector 100 comprises an injector body 110, an injector nozzle 120, a heating unit 140, and an actuation apparatus 150, in addition to strand of shape-memory material 130.

Injector body 110 can have any suitable configuration to facilitate direct or indirect manipulation of the injector 100 by surgical staff. Typically, the injector body will be sized and shaped to permit handling and manipulation using a single hand of an operator. The injector body may also maintain and/or be configured to permit operative coupling of the strand 130 (1) to apparatus for heating of the shape-memory material and (2) apparatus for actuation of the shape-memory material into an eye. In the illustrated embodiment, actuation apparatus 150 is maintained by the injector body and heating unit 140 is operatively coupled to the injector body.

Injector nozzle 120 is coupled to the injector body and facilitates entry of the injector into an incision in the cornea or sclera of an eye, and guides the shape-memory material into the eye. The injector nozzle has a lumen L extending therethrough and ends in an open tip 122 having a hole through which the shape-memory material exits the injector. The lumen may extend into the injector body. The shape of the nozzle and tip can be selected to permit entry through a relatively small incision (e.g., less than 3 mm) and to permit exit of the strand from the injector in a controlled manner to avoid damage to the eye. For example, the tip can be tapered and have a maximum outer dimension of less than 3 mm.

Prior to ejection from the injector, typically, strand of shape-memory material 130 is at least partially disposed in lumen L. Strand 130 is adapted to achieve a predetermined shape S when ejected from the open tip. For example, the shape memory material may be a material comprising nitinol.

Typically the shape to be achieved upon ejection is a closed shape (e.g., a circle, oval or polygon) or a substantially closed shape (e.g., greater than 75% of the circumference of a closed shape). It will be appreciated that a closed shape permits greater definition of the capsulotomy tear line along which the capsular bag will be torn to perform the capsulotomy. However, a non-closed shape may be used to partially define a tear line along which a rhexis is to be formed.

Heating unit 140 is coupled to the strand to cause the strand to achieve an elevated temperature. The heating unit may have any suitable configuration to permit the strand to reach an elevated temperature. For example, the heating unit may comprise a monopolar heating system. The heating unit may provide heat to the strand or may cause the strand to be heated (e.g., by providing electricity to the strand to generate heat).

The strand can be caused to be ejected from the open tip using any manual or automated technique. In the embodiment shown in FIG. 1A, actuation apparatus 150 is configured to cause the strand to be ejected from the open tip. The apparatus 150 may comprise a bar 154 connected to the strand to push the strand from the injector. In the illustrated embodiment, a bar is connected to the strand, and sliding the bar in a slot 152 pushes the strand from open tip 122. Alternatively, for example, apparatus 150 may comprise a button to activate an electromechanical apparatus that causes the strand to be ejected from open tip 122.

FIGS. 1B and 1C are schematic side and top view illustrations, respectively, of the injector shown in FIG. 1A in which the strand of shape-memory material has been ejected from the nozzle of the injector by operation of the actuation apparatus 150. As shown, the shape memory material forms a predetermined shape at a selected distance from open tip 122.

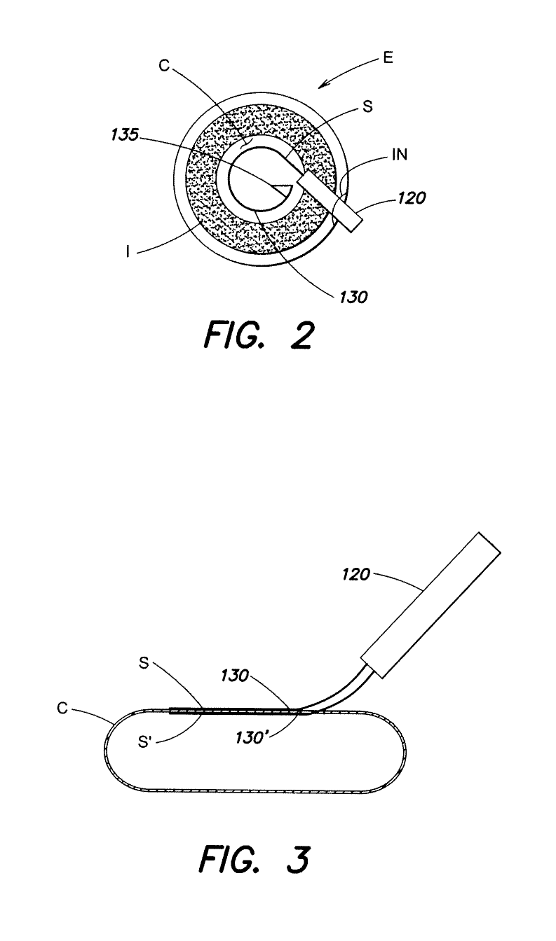

FIG. 2 is a schematic, partial, top view illustration of the injector shown in FIG. 1A in which the strand of shape-memory material has been ejected from the nozzle of the injector and into an incision I in an eye E on which a capsulotomy is to be performed. The shape-memory material forms the predetermined shape and is manipulated onto a selected surface of the eye's capsular bag. Typically activation of the heating unit occurs only after the shape-memory material is located onto the selected surface of the capsular bag C through iris I; however, prior activation is possible. Additionally, typically, the heating unit is deactivated and the strand retracted into the injector prior to removal of the injector from the eye.

The surface contacted and heated by the strand may be an interior or an exterior surface of the capsular bag. It will be appreciated that it may be appropriate to provide a surgeon with multiple devices each capable of forming a different predetermined shape S (e.g., circles of different diameters).

In some embodiments, strand 130 may be provided with a sharp tip 135 which, during the procedure of ejecting or retracting the strand from/into the injector, can cut or partially cut the capsular bag to further facilitate formation of the rhexis.

FIG. 3 is a schematic side view illustration of a second example of an injector for performing capsulotomies according to aspects of the present invention in which two strands of shape-memory material 130 and 130′ have been ejected from the nozzle of the injector. In the present embodiment, one strand is positioned onto an exterior surface of the capsular bag and operates substantially as described above, and strand 130′ is positioned on an interior surface of the capsular bag C. A purpose for having two strands is that the strands provide counter forces for each other thereby permitting more-intimate contact between strands 130 and 130′ and the capsular bag.

Strand 130 may be have the same shape as strand 130 or a different shape. Additionally, either one or both of strands 130 and 130′ may be heated. In the event that both strands are to be heated, heat may be caused by a single heating unit for both strands or separate heating unit for each strand. In some embodiments, it may be preferable to only heat the strand in contact with the inner surface of the capsular bag to reduce the likelihood of damaging the endothelial cells of the cornea.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An injector for performing a capsulotomy on a capsular bag, comprising:
an injector body;
an injector nozzle coupled to the injector body, the injector nozzle having a lumen extending therethrough ending in a tip having an opening;
a strand of shape-memory material adapted to achieve a predetermined shape when ejected from through opening; and
a heating unit coupled to the strand to cause the strand to achieve an elevated temperature.

2. The injector of claim 1, further comprising an actuation apparatus for causing the strand to be ejected through the opening.

3. The injector of claim 1, wherein the tip is adapted to fit through an incision in the eye of less than 3 mm.

4. The injector of claim 1, wherein the strand is adapted to form a closed shape or substantially closed shape upon ejection through the opening.

5. The injector of claim 1, wherein the shape-memory material comprises nitinol.

6. The injector of claim 1, wherein the heating unit comprises a monopolar heating system.

7. The injector of claim 1, wherein the strand is provided with a sharp tip to facilitate cutting of the capsular bag.

8. The injector of claim 1, wherein the injector comprises a second strand of shape-memory material.
